(19) United States
(12) Patent Application Publication
(10) Pub. No.: US 2008/0051774 A1
(43) Pub. Date: Feb. 28, 2008

(54) Device and method for coordinated insertion of a plurality of cryoprobes

(75) Inventors: Gil Ofir, Even-Yehuda (IL); Yaron Hefetz, Herzlia (IL); Mordechai Bliweis, Kiryat-Haim (IL)

Correspondence Address: Martin D. Moynihan;PRTSI, Inc., P.O. Box 16446, Arlington, VA, 22215, US

(73) Assignee: Galil Medical Ltd., Yokneam (IL)

(21) Appl. No.: 11/892,127

(22) Filed: Aug. 20, 2007

Related U.S. Application Data
(63) Continuation-in-part of application No. PCT/IL2007/000091, filed on Jan. 25, 2007.
Application No. PCT/IL2007/000091 is a continuation-in-part of application No. 11/637,095, filed on Dec. 12, 2006.
Application No. 11/637,095 is a continuation-in-part of application No. 10/660,478, filed on Sep. 12, 2003, now Pat. No. 7,150,743.
Application No. 10/660,478 is a continuation of application No. 09/860,486, filed on May 21, 2001, now Pat. No. 6,706,037.
Application No. 11/637,095 is a continuation-in-part of application No. 11/055,597, filed on Feb. 11, 2005.
Application No. 11/055,597 is a continuation of application No. 09/987,689, filed on Nov. 15, 2001, now abandoned.
Application No. 09/987,689 is a continuation of application No. 09/860,486, filed on May 21, 2001, now Pat. No. 6,706,037.
Application No. 11/637,095 is a continuation-in-part of application No. 11/185,699, filed on Jul. 21, 2005, now abandoned.
Application No. 11/185,699 is a division of application No. 10/151,310, filed on May 21, 2002, now abandoned.
Application No. PCT/IL2007/000091 is a continuation-in-part of application No. 11/640,309, filed on Dec. 18, 2006.
Application No. 11/640,309 is a continuation-in-part of application No. 10/660,478, filed on Sep. 12, 2003, now Pat. No. 7,150,743.
Application No. 10/660,478 is a continuation of application No. 09/860,486, filed on May 21, 2001, now Pat. No. 6,706,037.

(51) Int. Cl. A61B 18/02

(52) U.S. Cl. 606/20

(57) ABSTRACT

The present invention is of a cryotherapy apparatus which provides multiple cryoprobe operating tips in a configuration which enhances flow of exhaust gasses and thereby facilitates achieving cold temperatures and high thermal transfer capacity. The apparatus comprises a plurality of operating tips insertable in a body, each with a distal gas input lumen, a cryogen expansion chamber and a distal gas exhaust lumen, and a medial portion also insertable in a body and which comprises a medial gas exhaust lumen formed as a manifold communicating with the plurality of distal gas exhaust lumens.

### RELATED APPLICATIONS

This application is a continuation-in-part of PCT Patent Application No. PCT/IL2007/000091 filed Jan. 25, 2007, which is a continuation-in-part of pending U.S. patent application Ser. No. 11/637,095 filed Dec. 12, 2006, which is a continuation-in-part of U.S. patent application Ser. No. 10/660,478 filed Sep. 12, 2003, now U.S. Pat. No. 7,150,743, which is a continuation of U.S. patent application Ser. No. 09/860,486 filed May 21, 2001, now U.S. Pat. No. 6,706,037, which claims the benefit of U.S. Provisional Patent Application No. 60/242,455 filed Oct. 24, 2000, now expired.

U.S. patent application Ser. No. 11/637,095 is also a continuation-in-part of pending U.S. patent application Ser. No. 11/055,597 filed Feb. 11, 2005, which is a continuation of U.S. patent application Ser. No. 09/987,689 filed Nov. 15, 2001, now abandoned, which is a continuation-in-part of U.S. patent application Ser. No. 09/860,486 filed May 21, 2001, now U.S. Pat. No. 6,706,037, which claims the benefit of U.S. Provisional Patent Application No. 60/242,455, filed Oct. 24, 2000.

U.S. patent application Ser. No. 11/637,095 is also a continuation-in-part of U.S. patent application Ser. No. 11/185,699 filed Jul. 21, 2005, now abandoned, which is a divisional of U.S. patent application Ser. No. 10/151,310 filed May 21, 2002, now abandoned, which claims the benefit of U.S. Provisional Patent Application No. 60/300,097 filed Jun. 25, 2001, now expired, and U.S. Provisional Patent Application No. 60/291,990 filed May 21, 2001, now expired.

U.S. patent application Ser. No. 11/637,095 also claims the benefit of U.S. Provisional Patent Application No. 60/762,110 filed Jan. 26, 2006, now expired.

U.S. patent application Ser. No. 11/637,095 further claims the benefit of U.S. Provisional Patent Application No. 60/750,833 filed Dec. 16, 2005, now expired.

PCT Patent Application No. PCT/IL2007/000091 filed Jan. 25, 2007 is also a continuation-in-part of pending U.S. patent application Ser. No. 11/640,309 filed Dec. 18, 2006, which is a continuation-in-part of U.S. patent application Ser. No. 10/660,478 filed Sep. 12, 2003, now U.S. Pat. No. 7,150,743, which is a continuation of U.S. patent application Ser. No. 09/860,486 filed May 21, 2001, now U.S. Pat. No. 6,706,037, which claims the benefit of U.S. Provisional Patent Application No. 60/242,455 filed Oct. 24, 2000, now expired.

This application is also being filed concurrently with U.S. National Phase patent application Ser. No. ______ filed ______, titled “DEVICE AND METHOD FOR COORDINATED INSERTION OF A PLURALITY OF CRYOPROBES” (Attorney Docket No. 31852).

This application is also being filed concurrently with U.S. continuation-in-part (CIP) patent application Ser. No. ______ filed ______, titled “DEVICE AND METHOD FOR COORDINATED INSERTION OF A PLURALITY OF CRYOPROBES” (Attorney Docket No. 37192).

This application is also being filed concurrently with U.S. continuation-in-part (CIP) patent application Ser. No. ______ filed ______, titled “THIN UNINSULATED CRYOPROBE AND INSULATING PROBE INTRODUCER” (Attorney Docket No. 37193).

The contents of all the above-mentioned applications are incorporated herein by reference.

### FIELD AND BACKGROUND OF THE INVENTION

The present invention relates to devices and methods for thermal ablation of a surgical target within a body of a patient. More particularly, the present invention relates to a cryoablation device with improved thermal performance suitable for ablating a large target.

Cryoprobes cooled by Joule-Thomson are a generally preferred form of cryoprobe in many clinical contexts. These are cryoprobes which cool by expansion of a high-pressure cooling gas such as argon to a low-pressure state, resulting in rapid cooling of the expanding gas. High-pressure cooling gas is typically supplied through a high-pressure gas input lumen which delivers high-pressure cooling gas through a probe shaft to a distal probe operating tip. Cooling gas expansion typically takes place within the operating tip, when gas from the high-pressure gas input lumen transits a Joule-Thomson orifice into an expansion chamber. High-pressure cooling gas expands as it enters the expansion chamber and is thereby cooled. Some high-pressure gas may liquefy as a result of the expansion. Cold expanded cooling gas cools external walls of the expansion chamber, which walls in turn cool body tissues adjacent thereto. Liquefied gas (if any) within the expansion chamber will tend to evaporate, further cooling chamber walls and body tissues. Cold expanded and/or evaporated cooling gas is then typically exhausted from the expansion chamber through a gas exhaust lumen in the cryoprobe shaft, which lumen conducts the low-pressure cold expanded gas to atmosphere or, more rarely, to a gas collection system.

To achieve very low temperatures desirable for efficient cryoablation, Joule-Thomson cryoprobes generally comprise a heat-exchanger (also referred to herein as a “heat exchanging configuration”) to pre-cool high-pressure cooling gas prior to expansion. Cooling gas pre-cooled prior to expansion reaches extremely low temperatures after expansion. In prior art cryoprobes, a heat exchanger to accomplish such pre-cooling is typically positioned so as to facilitate heat transfer between gas input lumen and gas exhaust lumen, resulting in transfer of heat from relatively warm (e.g. room temperature) high-pressure cooling gas supplied in the cryoprobe gas input lumen to cold expanded cooling gas which, after exhausting from an expansion chamber in the probe's operating tip, transits the probe's gas exhaust lumen. Heat exchangers are typically constructed of highly thermally conductive materials such as metals and may include physical features designed to increase the efficiency of heat exchange, such as fins serving to increase the surface area over which heat is exchanged. For efficient heat transfer, heat exchangers typically provide a large surface of contact between a gas input lumen and a gas exhaust lumen, thereby enhancing thermal transfer from gas in the one lumen to gas in the other. Various configurations are used to enhance thermal transfer, but the need to provide a large surface of contact generally results in relatively thick and bulky construction, thereby limiting thinness and flexibility of cryoprobes in which they are used. Alternatively, very small heat exchangers may be used, but their thermal efficiency, and consequently their usefulness, is thereby limited.

Another factor limiting miniaturization of cryoprobes is the fact that temperature at the operating tip of a cryoprobe is limited by the evaporation temperature of the liquefied gas at the gas pressure of the gas within that tip. A thin gas exhaust lumen restricts gas output flow, causing increased “back pressure” and raising the evaporation temperature, resulting in higher tip operating temperatures and reduced cryoablation performance.

Cryosurgery is now used to treat a variety of clinical conditions. The present application is particularly relevant to cryosurgical treatment and/or cryoablation of large targets, such as large tumors or other large lesions. Yet the volume of assured tissue destruction around a cooling cryoprobe, and particularly around the highly miniaturized cryoprobes currently preferred in most clinical contexts, is generally limited to a volume of about a centimeter in diameter (within an iceball roughly two centimeters in diameter). Sequential repeated use of a single probe to sequentially ablate a series of segments of a large target, one segment after another, is generally too inefficient to be practical. Therefore, ablation of large targets generally requires simultaneous use of a plurality of probes.

Accordingly, a variety of patents and patent applications have taught devices and methods facilitating organized delivery of a plurality of cryoprobes to a large cryoablation target. For example, U.S. Pat. No. 6,706,037 to Zvuloni et al. presents an introducer having a hollow and a distal portion, the distal portion being sufficiently sharp so as to penetrate into a body, the hollow of the introducer being designed and constructed for containing a plurality of cryoprobes each of the cryoprobes being for effecting cryoablation, such that each of the plurality of cryoprobes is deployable through the distal portion of the introducer when the distal portion is positioned with respect to a tissue to be cryoablated. U.S. Patent Application 2006/0264920 by Duong also teaches a cryosurgical probe assembly including “a plurality of cryoprobes each having a shaft . . . , each shaft being deployable through a respective cryoprobe opening during operation to a deployed position used for ablating tissue.”

Zvuloni and Duong provide devices and methods for cryoablation of large cryoablation targets within a body, and are useful when target volume exceeds the volume which can be reliably cooled to cryoablation temperatures by a single inserted cryoprobe. By delivering a plurality of probes in a desired configuration to an ablation target, Zvuloni's “introducer” and Duong's “probe assembly” and their associated plurality of cryoprobes can be used to ablate targets too large to be ablated by a single probe.

Note is taken of PCT Application: IL2007/000091, filed Jan. 25, 2007, which teaches use of pre-bent cryoprobes useable to create a divergent array of cryoprobe operating tips when a plurality of cryoprobes extends distally from a cryoprobe introducer. PCT Application IL2007/000091 also teaches use of divergent cryoprobe channels within an introducer, useable to cause a plurality of flexible cryoprobes to diverge as they extend distally from a cryoprobe introducer. PCT Application IL2007/000091 is incorporated herein by reference.

### SUMMARY OF THE INVENTION

The probe-guiding mechanisms taught by Duong and Zvuloni are used to guide to their targets a plurality of separate and essentially independent probes. These and all similar arrangements have in common certain practical physical limitations common to all such coordinated uses of individual probes, which limitations constitute an upper limit on the efficiency of the cooling processes used to perform ablation by these prior art configurations. Thus, there is a widely recognized need for, and it would be highly advantageous to have, devices and methods enabling to deliver a plurality of cryotherapeutic operating tips to a voluminous treatment target, absent the physical limitations of these prior art configurations.

The present invention successfully addresses the shortcomings of these and other presently known configurations by providing a cryoablation apparatus having multiple cryoprobe operating tips configured for simultaneous ablation of a voluminous ablation target, the operating tips contiguous to a common shaft insertable within a body and comprising a common gas exhaust lumen. Embodiments of the present invention show improved thermal efficiency as compared to use of a plurality of individual probes, with or without a common introducer, as known to prior art.

The present invention is of a cryotherapy apparatus comprising a shaft which comprises at least one gas input lumen and a gas exhaust lumen, a first distal section distal to said shaft and which comprises an exhaust gas manifold, and a plurality of second distal sections distal to said first distal section, each of said second distal sections comprises a Joule-Thomson orifice and an expansion chamber. Alternatively, a distal portion of the gas exhaust lumen of the shaft may itself serve as “first distal section” comprising the exhaust gas manifold.

High-pressure cooling gas supplied through said gas input lumen expands within and cools the plurality of second sections, exhausts from those second sections to the manifold, then exhausts from the manifold through a common gas exhaust lumen in a common shaft. As is well known in the use of Joule-Thomson cryoprobes, an electrical heater or a heating gas such as helium may be also used as needed to heat the plurality of second distal sections. Thus, the apparatus may be thought of as a multi-headed cryoprobe providing significant advantages of thermal efficiency, more rapid cooling and colder achievable temperatures for a given probe diameter, and which is well adapted to cryoablation of voluminous ablation targets.

According to one aspect of the present invention there is provided a cryotherapy apparatus comprising a distal portion insertable in a body, the distal portion comprises a plurality of operating tips, each comprises a distal cryogen input lumen and a distal cryogen expansion chamber; and a medial portion which comprises a medial cryogen input lumen communicating with at least one of the distal cryogen input lumens and a medial cryogen exhaust lumen communicating with at least two of the distal cryogen expansion chambers.

According to further features in preferred embodiments of the invention described below, at least one of the operating tips comprises a Joule-Thomson cooler. The apparatus may comprise a medial heat exchanger operable to facilitate exchange of heat between the medial cryogen input lumen and the medial cryogen exhaust lumen.

According to further features in preferred embodiments of the invention described below, at least one of the operating tips further comprises a distal cryogen exhaust lumen and a distal heat exchanger which facilitates heat exchange between the distal cryogen exhaust lumen and the distal cryogen input lumen.

The medial portion is preferably configured for insertion in a body, and the apparatus may further comprise a proximal portion sized and configured to remain outside a body when the distal portion is inserted in a body. The proximal portion may comprise a first proximal cryogen input lumen which communicates with the medial cryogen input lumen, a proximal cryogen exhaust lumen which communicates with the medial cryogen exhaust lumen; and a heat exchanger which facilitates exchange of heat between the proximal cryogen input lumen and the proximal cryogen exhaust lumen.

The proximal portion may comprise a second proximal cryogen input lumen distinct from the first proximal cryogen input lumen, and a cryocooler communicating with the second proximal cryogen input lumen. The cryocooler may be a Joule-Thomson cooler.

According to further features in preferred embodiments of the invention described below, at least one of the operating tips is so configured that the distal gas input lumen is formed as a conduit within the distal exhaust lumen and the conduit is substantially straight, and is preferably also substantially flexible.

The outer diameter of at least one of the operating tips preferably less than 1.0 mm., more preferably less than 0.7 mm., and even more preferably less than 0.5 mm.

According to further features in preferred embodiments of the invention described below, the apparatus comprises a space-consuming heat exchanger only in the medial portion, or only in the proximal portion, or only in both medial and proximal portions.

According to further features in preferred embodiments of the invention described below, the distal cryogen input lumen and the distal cryogen exhaust lumen are uncoiled and are of substantially similar length.

According to further features in preferred embodiments of the invention described below, the medial cryogen input lumen is positioned within the medial cryogen exhaust lumen and is substantially straight.

According to further features in preferred embodiments of the invention described below, the medial portion does not contain a space-consuming heat exchanger.

According to further features in preferred embodiments of the invention described below, the medial cryogen input lumen is operable to transport high-pressure gas to at least two of the distal gas input lumens.

The apparatus may comprise a plurality of medial cryogen input lumens each operable to transport high-pressure gas to a one of the plurality of distal cryogen input lumens, and may comprise a plurality of heat exchangers, each operable to facilitate transfer of heat between input cryogen in one of the medial cryogen input lumens and exhaust cryogen within the medial cryogen exhaust lumen.

According to further features in preferred embodiments of the invention described below, a least a section of a lateral wall of the medial portion is insulated and a distal face of the medial portion is uninsulated.

According to further features in preferred embodiments of the invention described below, the apparatus is so configured that high-pressure input gas provided at a Joule-Thomson orifice within at least a one of the plurality of operating tips will undergo a first expansion within the operating tip and a subsequent second expansion when passing from the operating tip into the medial portion.

According to further features in preferred embodiments of the invention described below, at least one of the operating tips is pre-bent. Preferably, a plurality of the operating tips are pre-bent, and bends of the plurality of pre-bent operating tips are so oriented that distal ends of the operating tips diverge from each other when the operating tips are unconstrained. The apparatus may further comprise a tip-guiding sheath sized to accommodate the distal portion and at least a part of the medial portion, which sheath is operable to limit divergence of the distal ends of the operating tips when the distal portion is contained within the sheath.

The apparatus may further comprise a tip insertion guide having a plurality of outwardly diverging tip channels.

According to a further aspect of the present invention there is provided a cryotherapy apparatus which comprises a plurality of distal operating tips connected to a common medial shaft.

According to further features in preferred embodiments of the invention described below, the common shaft comprises at least one shaft subdivider which at least partially subdivides the medial shaft into a plurality of gas exhaust lumens.

According to a further aspect of the present invention there is provided a method of treating an organic cryotherapy target within a body of a patient, which method comprises providing a cryotherapy apparatus which comprises a distal portion insertable in a body, the distal portion comprises a plurality of operating tips, each comprises a distal cryogen input lumen and a distal cryogen expansion chamber; and a medial portion which comprises a medial cryogen input lumen communicating with at least one of the distal cryogen input lumens and a medial cryogen exhaust lumen communicating with at least two of the distal cryogen expansion chambers, inserting the plurality of operating tips into the organic cryotherapy target within a body of a patient, and supplying a cryogen to the plurality of expansion chambers via the medial and distal cryogen input lumens, thereby cooling the plurality of distal operating tips, thereby treating the cryotherapy target.

According to a further aspect of the present invention there is provided a method of cryoablation comprising providing a cryotherapy apparatus which comprises a plurality of operating tips and a common cryogen exhaust lumen, and supplying high-pressure gas to at least two of the operating tips.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a simplified schematic illustrating basic principles of operation of a Joule-Thomson cryoprobe, according to methods of prior art;

FIG. 2 is a simplified schematic illustrating basic principles of operation of a cryoprobe introducer, according to methods of prior art;

FIG. 3 is a simplified schematic showing a detail view of a portion of the introducer of FIG. 2, showing details of construction of an optional Joule-Thomson heater/cooler within the introducer, according to methods of prior art;

FIG. 4 is a simplified schematic of a multi-headed (i.e. multi-tipped) cryoablation apparatus, according to an embodiment of the present invention;

FIGS. 5a and 5b are simple schematics enable to compare the total gas-return lumen cross-section of a plurality of individual cryoprobes contained in the introducer of FIG. 2, according to methods of prior art, as shown in FIG. 5a, with the cross-section of a common gas return lumen of the apparatus of FIG. 4, according to an embodiment of the present invention, as shown in FIG. 5b;

FIG. 6 is a simplified schematic of a multi-tipped cryoablation apparatus having a plurality of operating tips without space-consuming heat exchanger, according to an embodiment of the present invention;

FIG. 7 is a simplified schematic of a multi-tipped cryoablation apparatus comprising an optional proximal pre-cooling unit, according to an embodiment of the present invention;

FIG. 8 is a simplified schematic presenting a multi-tipped cryoablation apparatus without proximal heat exchanger, according to an embodiment of the present invention;

FIGS. 9a-9d are simplified schematics of alternate configurations of a multi-tipped cryoablation apparatus having multiple independent gas input conduits, according to embodiments of the present invention;

FIG. 10 is a simplified schematic of a distal portion of a multi-tipped cryoablation device, according to an embodiment of the present invention;

FIG. 11 is a simplified schematic of a multi-tipped apparatus having pre-bent operating tips, combined with a tip-guiding sheath, according to an embodiment of the present invention;

FIG. 12 is a simplified schematic showing the apparatus of FIG. 11 at an advanced position within a tip-guiding sheath, according to an embodiment of the present invention;

FIG. 13 is a simplified schematic of a multi-tipped cryoablation apparatus with a tip insertion guide having a plurality of outwardly diverging tip channels, according to an embodiment of the present invention; and

FIGS. 14a and 14b are simplified schematics illustrating optional uses for heat insulation in multi-headed cryoprobes, according to embodiments of the present invention.

### DESCRIPTION OF THE PREFERRED EMBODIMENTS

The present invention relates to devices and methods for thermal ablation of a surgical target within a body of a patient. Specifically, the present invention can be used to deliver a plurality of cryoprobe operating tips to a cryoablation target and there provide highly efficient cryoablative cooling of those operating tips.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

To enhance clarity of the following descriptions, the following terms and phrases will first be defined:

The phrases “heat exchanger” and “heat-exchanging configuration” are used herein to refer to component configurations traditionally known as “heat exchangers”, namely configurations of components situated in such a manner as to facilitate the passage of heat from one component to another. Examples of “heat-exchanging configurations” of components include a porous matrix used to facilitate heat exchange between components, a structure integrating a tunnel within a porous matrix, a structure including a coiled conduit within a porous matrix, a structure including a first conduit coiled around a second conduit, a structure including one conduit within another conduit, or any similar structure.

The phrase “Joule-Thomson heat exchanger” as used herein refers, in general, to any device used for cryogenic cooling or for heating, in which a gas is passed from a first region of the device, wherein it is held under higher pressure, to a second region of the device, wherein it is enabled to expand to lower pressure. A Joule-Thomson heat exchanger may be a simple conduit, or it may include an orifice, referred to herein as a “Joule-Thomson orifice”, through which gas passes from the first, higher pressure, region of the device to the second, lower pressure, region of the device. A Joule-Thomson heat exchanger may further include a heat-exchanging configuration, for example a heat-exchanging configuration used to cool gasses within a first region of the device, prior to their expansion into a second region of the device.

The phrase “cooling gasses” is used herein to refer to gasses which have the property of becoming colder when expanded through a Joule-Thomson heat exchanger. As is well known in the art, when gasses such as argon, nitrogen, air, krypton, CO₂, CF₄, and xenon, and various other gasses, at room temperature or colder, pass from a region of higher pressure to a region of lower pressure in a Joule-Thomson heat exchanger, these gasses cool and may to some extent liquefy, creating a cryogenic pool of liquefied gas. This process cools the Joule-Thomson heat exchanger itself, and also cools any thermally conductive materials in contact therewith. A gas having the property of becoming colder when passing through a Joule-Thomson heat exchanger is referred to as a “cooling gas” in the following.

The phrase “heating gasses” is used herein to refer to gasses which, when passed at room temperature or warmer through a Joule-Thomson heat exchanger, have the property of becoming hotter. Helium is an example of a gas having this property. When helium passes from a region of higher pressure to a region of lower pressure, it is heated as a result. Thus, passing helium through a Joule-Thomson heat exchanger has the effect of causing the helium to heat, thereby heating the Joule-Thomson heat exchanger itself and also heating any thermally conductive materials in contact therewith. Helium and other gasses having this property are referred to as “heating gasses” in the following.

As used herein, a “Joule Thomson cooler” is a Joule Thomson heat exchanger used for cooling. As used herein, a “Joule Thomson heater” is a Joule Thomson heat exchanger used for heating. A Joule-Thomson heater/cooler is thus a “Joule-Thomson heat exchanger” as defined above.

The terms “ablation temperature” and “cryoablation temperature”, as used herein, relate to the temperature at which cell functionality and structure are destroyed by cooling. According to current practice temperatures below approximately −40° C. are generally considered to be ablation temperatures.

The term “ablation volume”, as used herein, is the volume of tissue which has been cooled to ablation temperature by one or more cryoprobes.

As used herein, the term “high-pressure” as applied to a gas is used to refer to gas pressures appropriate for Joule-Thomson cooling of cryoprobes. In the case of argon gas, for example, “high-pressure” argon is typically between 3000 psi and 4500 psi, though somewhat higher and lower pressures may sometimes be used.

The terms “thermal ablation system” and “thermal ablation apparatus”, as used herein, refer to any apparatus or system useable to ablate body tissues either by cooling those tissues or by heating those tissues.

For exemplary purposes, the present invention is principally described in the following with reference to an exemplary context, namely that of cryoablation of a treatment target by use of cryoprobes operable to cool tissues to cryoablation temperature. It is to be understood that invention is not limited to that exemplary context. The invention is, in general, relevant to cryogenic treatment of any surgical target by means of a plurality of operating tips utilizing a common conduit for exhausting cryogen from a vicinity of those operating tips. For simplicity of exposition, Joule-Thomson cryoprobes are presented in the Figures and reference is made to Joule-Thomson cryoprobes in description of preferred embodiments provided hereinbelow, yet all such references are to be understood to be exemplary and not limiting. Thus, discussion of Joule-Thomson cryoprobes hereinbelow may be understood to apply also to evaporative cryoprobes or cryoprobes of any other sort, so long as those probes require to exhaust used cryogen from their operating tips during or after use. Similarly, references to cryoablation of tissues are also to be understood as exemplary and not limiting. Thus, references to cryoablation are to be understood as referring also non-ablative cryogenic treatment of tissues.

It is expected that during the life of this patent many relevant cryoprobes will be developed, and the scope of the terms “cryoprobe” and “sheath” and “introducer” is intended to include all such new technologies a priori.

As used herein the term “about” refers to ±10%.

In discussion of the various figures described hereinbelow, like numbers refer to like parts. The drawings are generally not to scale. Some optional parts are drawn using dashed lines.

For clarity, non-essential elements are omitted from some of the drawings.

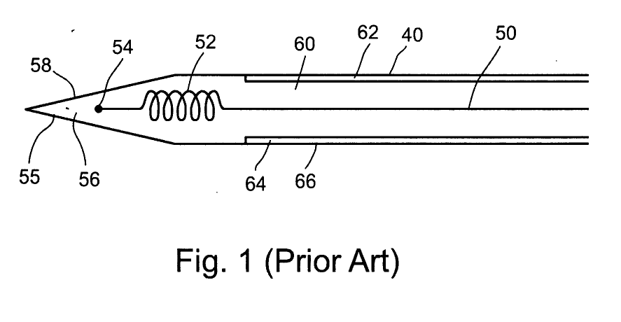

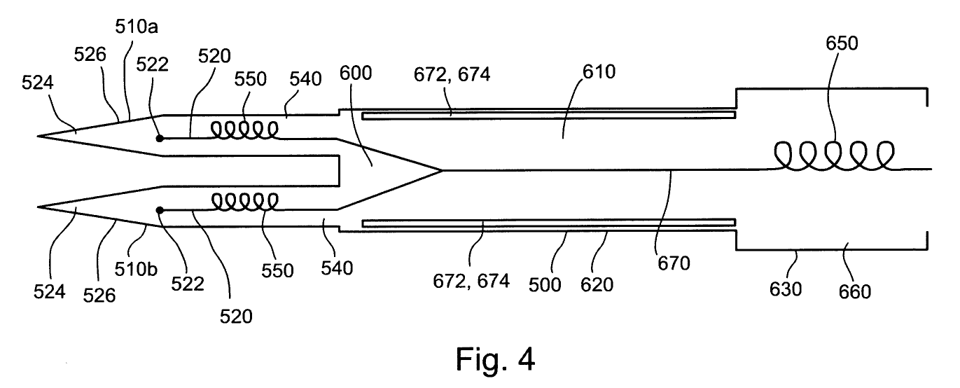

For purposes of better understanding the present invention, as illustrated in FIGS. 4-13 of the drawings, reference is first made to the construction and operation of a conventional (i.e., prior art) cryoprobe/introducer combination, as illustrated in FIGS. 1-3.

Referring now to the drawings, attention is now drawn to FIG. 1 which is a simplified schematic which illustrates basic principles of operation of a Joule-Thomson cryoprobe, according to methods of prior art. FIG. 1 presents a cryoprobe 40 which comprises a high-pressure gas input lumen 50 which supplies high-pressure gas through a heat exchanger 52 to a Joule-Thomson orifice 54 within an operating tip 55. When cryoprobe 40 is operated in cooling, high-pressure cooling gas is supplied through gas input lumen 50. High-pressure cooling gas passes through Joule-Thomson orifice 54 into an expansion chamber 56 within operating tip 55, where that cooling gas expands. Rapid expansion of cooling gas in expansion chamber 56 cools the cooling gas, some of which may liquefy. Cold expanded gas cools walls 58 of operating tip 55, and thereby cools tissues adjacent to those walls. Liquefied gas (if any) within expansion chamber 56 may then evaporate, further cooling walls 58. Cold expanded and/or evaporated gas exhausts from operating tip 56 by passing through or over heat exchanger 52, and exhausts from probe 40 through gas exhaust lumen 60 which runs the length of shaft 66 of probe 40. Heat exchanger 52 serves to pre-cool high-pressure cooling gas supplied in gas input lumen 50 by facilitating heat exchange between input high-pressure gas (normally initially at room temperature) and cold expanded gasses exhausting from operating tip 56. Thermal insulation 62 or a heating element 64 may be provided to protect tissues adjacent shaft 66 from cold damage caused by cold temperatures induced in shaft 66 by cold cryogen (cold expanded gasses) exhausting from tip 56.

It is generally found useful for cryoprobes to provide heating capacity as well, to facilitate their displacement or removal from body tissues which adhere to the cryoprobe during freezing and cryoablation. In generally preferred embodiments, cryoprobe 40 as here described may simply connected to a source of high-pressure heating gas, which gas heats on expansion into chamber 56, resulting in heating of walls 58 and facilitating disengagement of probe 40 from frozen tissues. When heating gas is supplied, heat exchanger 52 serves to pre-heat that gas by facilitating exchange of heat between high-pressure input gas and hot expanded heating gas exhausting from operating tip 56. For efficient use, cryoprobe 40 is preferably connected to a gas supply module operable to supply either high-pressure heating gas or high-pressure cooling gas on demand.

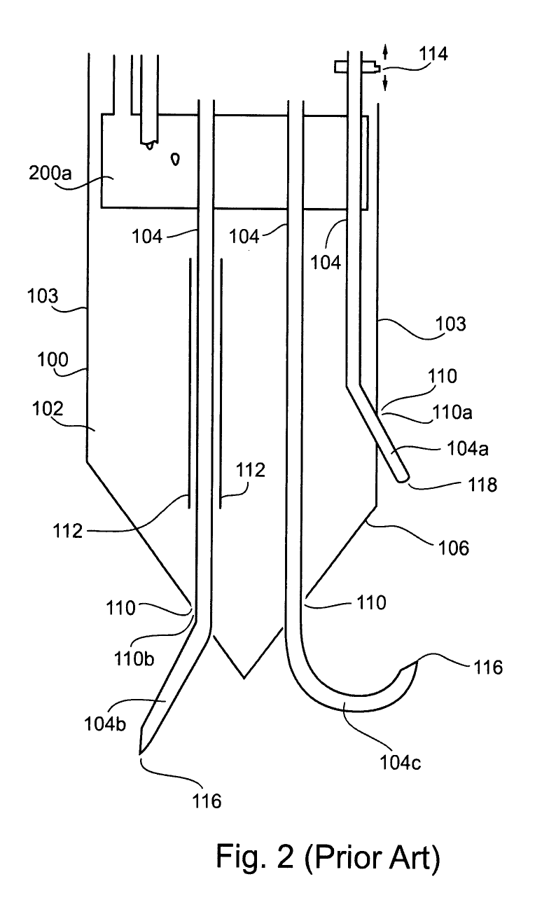

Attention is now drawn to FIG. 2, which is a simplified schematic illustrating basic principles of operation of a cryoprobe introducer, according to methods of prior art. An introducer 100 is optionally designed and constructed so as to be sufficiently sharp so as to easily penetrate through body tissue, inflicting minimal damage to nearby tissues. Introducer 100 has a hollow 102. Hollow 102 is designed and constructed for containing a plurality of cryoprobes 104. Cryoprobes 104 may be Joule-Thomson cryoprobes 40 as presented by FIG. 1, or evaporative cryoprobes, or any other form of cryoprobe requiring supply of cryogen to an operating tip through a cryogen input lumen and exhaustion of cryogen from that operating tip through a cryogen exhaust lumen. Each of cryoprobes 104 is capable of being cooled to a low temperature, say −60 to −120° C., or less, and is capable of freezing tissues, for effecting cryoablation.

A distal portion 106 of introducer 100 is formed with a plurality of openings 110. As is further detailed hereinbelow, openings 110 of introducer 100 serve for deployment therethrough of a plurality of cryoprobes 104. Each of cryoprobes 104 contained within introducer 100 is deployable outside introducer 100, and in the deployed state is capable of effecting cryoablation. Hollow 102 may optionally be partitioned into a plurality of longitudinal compartments 112, each compartment 112 is designed and constructed for containing at least one, preferably one, cryoprobe 104. Hollow 102 of introducer 100 optionally includes a Joule-Thomson heater/cooler 200a (also referred to as a “Joule-Thomson heat exchanger” in the definitions section hereinabove, and described in further detail hereinbelow) for pre-heating and pre-cooling at least a portion of hollow 102, thereby further pre-cooling cooling gasses (or pre-heating heating gasses) used for cooling (or heating) of cryoprobes 104. External sheath 103 of introducer 100 may include thermally insulating material(s), so as to prevent heat exchange between hollow 102 of introducer 100 and tissues of the body, when introducer 100 is introduced into a body.

The mode of operation of introducer 100 involves introducing introducer 100 with its plurality of cryoprobes 104 contained within hollow 102 into the body of a patient, then, deploying through openings 110 present at distal portion 106 of introducer 100 at least one of cryoprobes 104, and cooling the deployed cryoprobe or cryoprobes 104 to perform cryoablation.

The image of introducer 100 has been expanded in FIG. 2, so as to enhance visibility of details, yet introducer 100 is preferably thin in construction, so as to permit its introduction into the body in a manner that minimizes damage to tissues present along its penetration path, leading to the intended site of cryoablation. Preferably a cross-section of sheath 103 will not exceed 6 mm. In a recommended mode of operation, cryoprobes 104 are initially positioned within introducer 100 (i.e., retracted) so that they do not hinder the penetration of the introducer into the body of the patient. Each cryoprobe 104 is designed and constructed deployable through openings 110 present at distal portion 106 of introducer 100, when distal portion 106 is appropriately positioned with respect to a tissue to be cryoablated. An optional deploying and retracting member 114, shown in FIG. 2 as coupled to cryoprobe 104a, may be operably coupled to some or all of cryoprobes 104. For most applications it will be convenient for cryoprobes 104 to be provided with sharp distal ends 116 to aid in penetration of tissues during deployment, yet under some circumstances a cryoprobe 104 with a blunt or rounded distal end 118 may preferably be used.

In a recommended embodiment each of cryoprobes 104 has a cross section of between 0.3 mm and 3 mm. In their un-deployed, retracted, state, cryoprobes 104 will fit in the space made available for them within hollow 102 of introducer 100. This allows introducer 100 to penetrate the body of a patient with little hindrance. Once at the desired cryoablation site, some or all of cryoprobes 104 are deployed beyond introducer 100, penetrating further into the body's tissues, at which time cryoablation is performed.

In a preferred embodiment of the invention, cryoprobes 104 are designed and constructed to advance, during deployment, in a plurality of different directions. Generally, some of the cryoprobes are designed and constructed so as to expand laterally away from the introducer when deployed.

As cryoprobes 104 so designed and constructed advance from within introducer 100 and deploy in a lateral direction away from the periphery of introducer 100, they thereby define a three-dimensional cryoablation volume. When deployed cryoprobes 104 are cooled to cryoablation temperatures, e.g., −60° to −160° C., preferably −80° to −120° C., the cooled volumes provided by each of the deployed cryoprobes 104 combine to produce a shaped cooled volume within which cryoablation is effected. This method of arranging and deploying the cryoprobes thus enables to create a three-dimensional cryoablation volume of a predetermined size and shape.

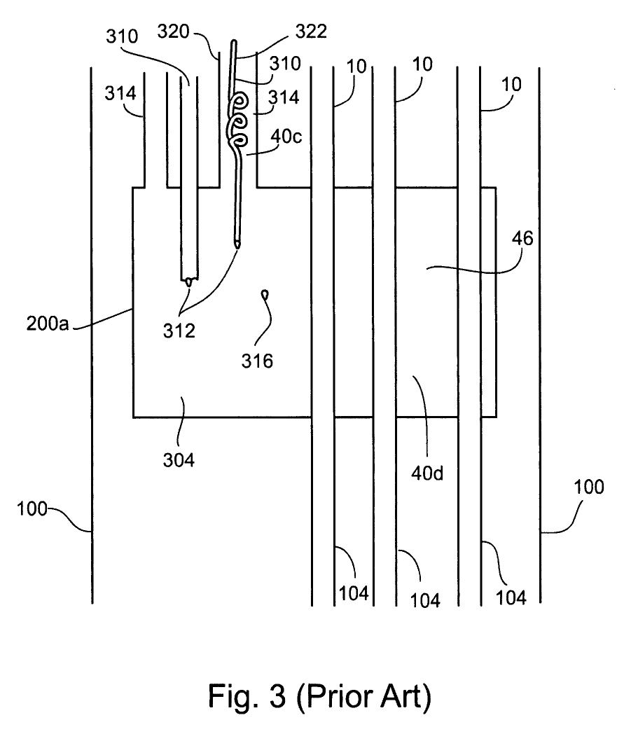

Attention is now drawn to FIG. 3, which is a simplified schematic of a portion of introducer 100, showing in particular further details of construction of optional Joule-Thomson heater/cooler 200a, according to methods of prior art.

FIG. 3 shows a detail view of a portion of an introducer 100. Passageways 10, which serve for passing gas from high-pressure gas sources outside introducer 100 to cryoprobes 104, are situated near or within a chamber 304 within hollow 102 of introducer 100. Gas input passageway 310 provides high-pressure cooling or heating gasses which pass from passageway 310 through Joule-Thomson orifice 312, and expand into chamber 304. Cooling gasses passing from passageway 310 through Joule-Thomson orifice 312 expand and are thereby cooled and may liquefy. Cooling gasses cooled by expansion, and a cryogenic pool of liquefied gasses which may form, cool chamber 304. Alternatively, heating gasses, which have an inversion temperature lower than the temperature obtained by liquefaction of the cooling gasses, pass from passageway 310 through Joule-Thomson orifice 312 and heat chamber 304. The gasses are subsequently exhausted to the atmosphere through passageway 314. Optional thermal sensor 316, which may be a thermocouple, monitors temperatures in chamber 304.

In a preferred embodiment, heat exchanger 200a includes a heat-exchanging configuration 40c for facilitating exchange of heat between incoming gasses entering heat exchanger 200a through passageway 310 and exhaust gasses being exhausted to the atmosphere through passageway 314 after passing through Joule-Thomson orifice 312. In this embodiment passageway 310 for incoming gasses and passageway 314 for exhaust gasses are constructed of heat conducting material, such as a metal, and are constructed contiguous to each other, or wrapped one around the other. In an alternative construction of heat-exchanging configuration 40c, passageway 314 is implemented as a porous matrix 320 through which expanded gasses are exhausted to atmosphere. In this construction, passageway 310 is implemented as conduit 322 for incoming gasses formed within porous matrix 320. Conduit 322 may be formed as a straight conduit tunneling through porous matrix 320, or it may be formed as a spiral conduit integrated with porous matrix 320.

Heating gasses being exhausted through passage 314 after having passed through Joule-Thomson orifice 312 are hotter than incoming heating gasses entering through passageway 310. Consequently, exchange of heat between passageway 310 and passageway 314 has the effect of preheating incoming heating gasses, thereby enhancing efficiency of the apparatus.

Similarly, cooling gasses being exhausted through passage 314 after having passed through Joule-Thomson orifice 312 are colder than incoming cooling gasses entering through passageway 310. Consequently, exchange of heat between passageway 310 and passageway 314 has the effect of pre-cooling incoming cooling gasses, thereby enhancing efficiency of the apparatus.

Passageways 10 are preferably made of a thermally conducting material, such as a metal. Consequently, heating or cooling chamber 304 pre-heats or pre-cools the gasses passing through passageways 10 towards cryoprobes 104. Thus, the arrangement here described constitutes a heat-exchanging configuration 40d, for facilitating exchange of heat between heating and cooling chamber 304 and gas passing through passageways 10. In an alternate construction, heat-exchange configuration 40d is formed by implementing a portion of passageways 10 as either straight or spiral conduits tunneling through a porous matrix 46 occupying a portion of chamber 304. In yet another alternative arrangement, cryoprobes 104 themselves pass through chamber 304, resulting a similar pre-heating or pre-cooling effect. Chamber 304 may also be designed and constructed such that heating and cooling of chamber 304 has the effect of heating and cooling all or most of hollow 102 of introducer 100, and, as a result, all or most of the contents thereof.

Configurations such as those shown in the prior art FIGS. 1-3 and discussed above, and those presented in prior art patents and patent applications discussed in the background section hereinabove, share a common disadvantage which is overcome in embodiments of the present invention. That common disadvantage may be explained as follows. Cryoprobes, whether inserted in a patient's body individually or inserted collectively by means of an introducer or other form of probe assembly, typically traverse healthy body tissue on the way to the lesion whose cryoablation is desired. Thin probes and introducers are desirable: the larger the cross-section of probes and/or introducers traversing healthy tissue, the greater the damage to that healthy tissue.

This problem is particularly important with respect to cryoablation of voluminous ablation targets, as such ablation typically requires simultaneous use of multiple probes, with attendant danger of multi-site damage to healthy tissue. Accordingly, preferred contemporary clinical practice has evolved a need for highly miniaturized cryoprobes, and such probes are highly preferred by clinicians. As minimally invasive endoscopic cryosurgery has become increasingly popular, the importance of cryoprobe miniaturization has become even greater: endoscopic cryoablation, enabling to approach and ablate lesions endoscopically in and around various organs of the body, is a highly preferred form of surgery, and endoscope approaches require significant cryoprobe miniaturization.

Highly miniaturized cryoprobes, however, along with their undoubted advantages of small size, have an important double disadvantage. As explained above, Joule-Thomson probes depend for their functioning on supply of high-pressure gas to their operating tips, which gas is supplied through a narrow high-pressure conduit. As diameter of such conduits is reduced, the amount of gas which can be supplied therethrough, even at high pressure, is necessarily reduced also. Yet limitation in the amount of cooling gas which can be supplied through a probe's gas input lumen limits the amount of cooling which can be accomplished by that probe. Equally important, Joule-Thomson probes depend for their functionality on rapid and efficient evacuation of expanded gas from probes' operating tips. Indeed, the degree to which high-pressure gas expands and cools is a function of the pressure differential between pressure of high-pressure cooling gas supplied, and pressure within expansion chambers (such as chamber 56 discussed above) within their operating tips. Any reduction in that pressure differential results in a limitation in the degree of cooling achievable by the probe.

For example, most Joule-Thomson cryoprobes in clinical use today are powered with high-pressure argon supplied at pressure in the range of 200-300 atmospheres. Full expansion of that high-pressure gas (i.e. expansion down to one atmosphere of pressure) would result in cooling down to a temperature of around −186° C. Yet such expansion would require expansion of the gas into an extremely large volume, and no miniaturized probe can supply an evacuation conduit large enough to permit such radical expansion absent significant back-pressure in the gas evacuation conduit. In practice, real-time pressure measurements taken with prior art cryoprobes in contemporary use reveal back-pressures on the order of 50 atmospheres in Joule-Thomson expansion chambers and gas exhaust lumens of those probes. Similarly, since cooling effects of Joule-Thomson probes generally depends in part on evaporation of liquefied gas within operating tips, a controlling factor for temperature achievable by a probe is the evaporation temperature of the cooling gas (e.g. argon) at the pressure obtaining within the expansion chamber of the probe. Consequently, rather than achieving cooling temperature near a theoretical minimum of around −170° C., temperatures of only around −120° C. are more typical.

Thus, the degree of coldness which can be achieved in any given probe is a function of the flow capacity of the gas exhaust lumen of that probe: the greater the flow-capacity of the gas exhaust lumen, the lower the temperature that can be achieved for a given cooling gas at a given input pressure. Moreover, the higher the output flow rate, the higher the input flow rate of high-pressure gas that can be accommodated without creating undesired back pressure. Higher gas flow-through capacity translates to higher cooling capacity of the probe.

Thus, high flow-through rate and low backpressure in the gas exhaust lumen are highly desirable, yet difficult or impossible to achieve in highly miniaturized cryoprobes. Accordingly, embodiments of the present invention are provided to provide multiple cryoprobe operating tips operable to be delivered to a cryoablation target within a body and operable there to be inserted in that target and cooled to cryoablation temperatures, in a configuration which enhances flow of exhaust gasses and thereby facilitates achieving cold temperatures and high thermal transfer capacity of the apparatus.

Attention is now drawn to FIG. 4, which is a simplified schematic of a multi-tipped (i.e. multi-headed) cryosurgery apparatus, according to an embodiment of the present invention.

FIG. 4 presents a cryotherapy apparatus 500 having a plurality of operating tips 510, labeled 510a and 510b in the Figure. Operating tips 510 are collectively referred to as a “distal portion” of apparatus 500 in the claims hereinbelow.

Each operating tip 510 comprises an operating tip cryogen input lumen, here embodied as gas input lumen 520, a Joule-Thomson orifice 522 an expansion chamber 524, and a cryogen exhaust lumen, here embodied as a gas exhaust passage 540. It is noted that gas exhaust passage 540 need not necessarily be distinct from expansion chamber 524, and may simply be an extension thereof.

In an exemplary apparatus presented in FIG. 4 and in those presented in the following figures, two distinct operating tips labeled 510a and 510b are provided, but it is to be understood that this example is exemplary only and not intended to be limiting: three, four, or more distinct operating tips may be provided in additional preferred embodiments of the present invention.

In similarity to the exemplary Joule-Thomson cryoprobe 40 presented in FIG. 1, in each operating tip 510 high-pressure gas input lumen 520 supplies high-pressure gas to a Joule-Thomson orifice 522 within operating tip 510. When operating tip 510 is operated in cooling, high-pressure cooling gas is supplied through gas input lumen 520. High-pressure cooling gas passes through Joule-Thomson orifice 522 into an expansion chamber 524 within operating tip 510, where that cooling gas expands. Rapid expansion of cooling gas in expansion chamber 524 cools the cooling gas, some of which may liquefy. Cold expanded gas cools walls 526 of operating tip 510, and thereby cools tissues adjacent to those walls. Liquefied gas (if any) within expansion chamber 524 may then evaporate, further cooling walls 526. Cold expanded gas exhausts from each operating tip 510 through a relatively short tip exhaust passage 540.

A small operating tip heat exchanger 550 may be provided within passage 540 to pre-cool gas transiting input lumen 520, yet operating tip heat exchanger 550 is optional and may be omitted, as will be explained hereinbelow. In similarity to cryoprobe 40, in a recommended mode of operating tips 510 may be alternately supplied with cooling gas and with heating gas in gas input lumen 520. Supplying heating gas in gas input lumen 520 results in heating operating tips 510, enabling a surgeon to engage in repeated heating/cooling cycles and/or to heat operating tips 510 to facilitate disengagement of apparatus 500 from frozen tissues following treatment.

As may be seen in FIG. 4, apparatus 500 is characterized by a common gas exhaust lumen 610, also referred to herein as medial exhaust lumen 610. A distal portion of common gas exhaust lumen 610 functions as an exhaust gas manifold 600 (also referred to herein as medial portion 600) located near and preferably located proximally to operating tips 510 and communicating with operating tip exhaust passages 540. Gas exhaust manifold 600 receives exhaust gas from a plurality of operating tip exhaust passages 540, which gas is thence exhausted through common gas exhaust lumen 610 which runs the length of proximal shaft 620 of apparatus 500 and conducts exhaust gasses received from exhaust passages 540 to atmosphere, or optionally to a gas collection system and/or to a suction system. In the claims hereinbelow, a portion of apparatus 500 containing exhaust gas manifold 600 and that part of shaft 620 (and that part of gas exhaust lumen 610) which is insertable in a body are referred to herein as a “medial portion” of apparatus 500. An optional portion of apparatus 500 not insertable in a body, including an optional handle 630, is referred to as a “proximal portion” of apparatus 500 in the claims hereinbelow.

In a preferred embodiment presented in FIG. 4, a common gas input lumen 670 (also referred to herein as medial gas input lumen 670) in shaft 620 supplies high-pressure input gas to a plurality of distal operating tip gas input lumens 520. Thus, when apparatus 500 is operated in cooling, high-pressure cooling gas is supplied to expansion chambers in a plurality of operating tips and cools those tips, and expanded cooled gas exhausts from those tips into gas exhaust manifold 600 and is conducted from apparatus 500 through common gas exhaust lumen 610.

Optionally, as shown in FIG. 4, a common heat exchanger 650 may be supplied within common gas exhaust lumen 610. When apparatus 500 is operated in cooling, heat exchanger 650 serves to pre-cool high-pressure cooling gas transitioning heat exchanger 650 towards operating tip gas input lumens 520. Heat exchanger 650 serves to facilitate transfer of heat from high-pressure input cooling gas to cold expanded cooling gasses flowing through gas exhaust lumen 610 as they exhaust from apparatus 500. When apparatus 500 is operated in heating, heating gas transiting heat exchanger 650 is pre-heated by thermal transfer from hot expanded heating gas exhausting in gas exhaust lumen 610. Heat exchanger 650 may be positioned within a distal portion of common gas exhaust lumen 610, or alternatively may be positioned at a proximal portion of shaft 620, or in a wider proximal portion 660 of shaft 620, such as in a handle 630.

It is noted that FIG. 4, and other Figures discussed hereinbelow, present embodiments of the present invention implemented using Joule-Thomson cooling techniques. Thus, cryogen input conduits and often referred to as “gas input lumens” and cryogen exhaust conduits are referred to as “gas exhaust lumens”. It is to be understood that Joule-Thompson implementations of embodiments of the present invention are exemplary, and not intended to be limiting. Thus, gas input lumens are examples of cryogen input conduits. Optional alternative embodiments of the present invention may comprise cryogen evaporation systems wherein cryogen is presented as liquefied gas, is supplied substantially as a liquid through a cryogen supply lumen, expands into an expansion chamber by evaporation from the liquid state, and exhausts, as a gas following evaporation, through a common cryogen exhaust lumen 610 in shaft 620.

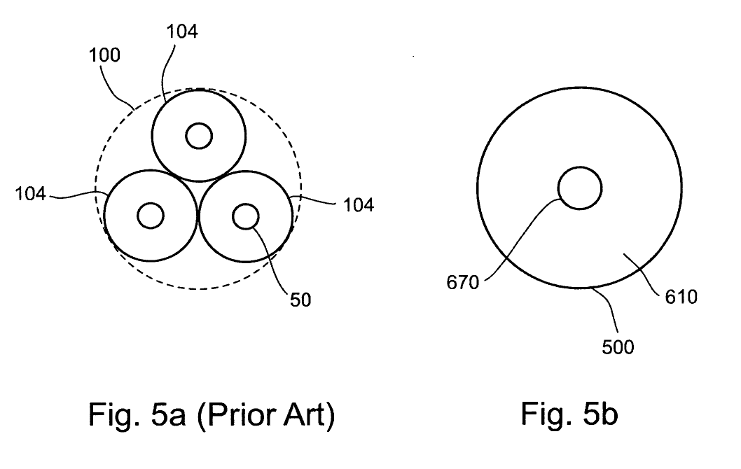

Attention is now drawn to FIGS. 5a and 5b, which are simple schematics enabling to compare the cross-section of common gas return lumen 610 of apparatus 500, shown in FIG. 5b, with the gas-return lumen cross-sections of a plurality of individual cryoprobes such as cryoprobes 104 contained in a common introducer such as introducer 100 presented by FIG. 2 and described hereinabove, shown in FIG. 5a.

As may easily be seen from FIGS. 5a and 5b, if apparatus 500 and introducer 100 have a same diameter, apparatus 500 presents a much larger cross-section for exhaust gas transmission. Actual flow characteristics of exhaust gas within an apparatus 500 of diameter D, as compared to flow characteristics of exhaust gas within cryoprobes 104 within an introducer 100 also of diameter D, cannot be simply described, as they depend on characteristics of the specific gasses used, gas pressures, conduit wall characteristics, positions and shapes of gas input sources (which positions and shapes influence induced turbulence in the flow), and so on. However it is obvious from inspection of FIGS. 5a and 5b that for a given same diameter, apparatus 500 clearly provides a larger cross-sectional area and less friction between moving gas and static walls than is provided by a set of probes 104 within an introducer 100 of that diameter.

Thus, apparatus 500, with its larger cross-section and reduced internal friction (for a same diameter) provides a more efficient pathway for exhaustion of used cryogen from the vicinity of operating tips 510, as compared to a set of cryoprobes within an introducer. Thus, for a given maximum instrument diameter, apparatus 500, as compared to a prior-art introducer plus cryoprobes, allows for significantly higher gas output flow.

Increased exhaust gas flow results in lower back pressure in the gas output portion of apparatus 500, as compared to the back pressure resulting in miniaturized prior-art probes. Reduced back pressure results in greater expansion of expanding gasses, as discussed above. Consequently, apparatus 500 can generally achieve temperatures at operating tips 510 that are lower than temperatures achievable by a set of prior art cryoprobes deliverable by an introducer 100 of comparable dimensions. Increased exhaust gas flow similarly increases gas throughput, enabling apparatus 500 to provide higher heat transfer than a comparable set of prior-art probes. Further, additional space volume available within shaft 620 of apparatus 500 as compared to a set of prior art probes in an introducer of comparable diameter enables to provide a gas input lumen 670 which also presents a larger cross-section and lower resistance than that made available by a set of gas input lumens 50 of a comparable set of cryoprobes 40/104 within an introducer 100 of comparable size. In sum, apparatus 500 can cool faster and colder than can a set of conventional cryoprobes delivered by an introducer 100 of comparable size.

It is an additional advantage of apparatus 500 that, rather than providing enhanced cooling and lower temperatures, an apparatus 500 can be designed to provide cooling characteristics similar to those of an introducer 100 with a plurality of included cryoprobes 104, in an apparatus 500 having a shaft which is thinner than that of the corresponding introducer 100.

An additional advantage of apparatus 500 is noted: in many clinical contexts it is important that all or part of cryoprobe shafts or introducer shafts be insulated, to prevent cold exhaust gasses from drawing excessive heat from healthy tissues surrounding those inserted shafts and thereby damaging the tissues. For this purpose, optional thermal insulation 672 and/or optional electrical or other heating elements 674 are provided in shaft 610 of apparatus 500. The greater amount of space available in shaft 670 as opposed to shafts of probes 104 and introducer 100 enables insulator 672 to be more bulky (and consequently more effective) than that which may practically be provided (given the requirements for high gas throughput discussed hereinabove) in probes 104 and introducer 100.

It is noted that whereas a circular cross-sectional shape of shaft 610 generally provides most efficient gas transport along the shaft, the generally high thermal efficiency of apparatus 500 as compared to prior art probes enables use of non-circular (e.g. oval) cross-sections without thereby excessively impeding gas flow through the shaft. Non-circular cross-sections may in some cases be considered preferable e.g. for minimizing damage to healthy tissues in certain clinical contexts.

A few simplified calculations may be considered to put the above considerations into a practical context. By rough approximation, without taking into account wall thickness and neglecting the (typically very thin) high pressure input tube, the common gas return shaft of FIG. 5b has a cross-sectional area approximately 1.5 times that provided by the three individual shafts shown in FIG. 5a. If we assume by way of example that each individual 104 probe is 1 mm in diameter, then the common shaft of FIG. 5b would be 2.1 mm in diameter. If we then assume an insulation layer 0.15 mm thick in each case, the resulting available diameters are 0.7 mm for the individual probe tubes and 1.8 mm for the common tube. This gives a cross-sectional ratio of 2.2 in favor of the common shaft of FIG. 5b. Moreover, since friction between moving gas and the walls of tubes within which the gas moves is a function of area of contact between gas and walls, and the ratio of wall surface to tube cross-section is smaller for larger tubes, relative restriction of gas flow in FIG. 5a prior-art devices may be expected to be even greater than 2.2 cross-sectional ratio implies. (These calculations are of course exemplary only. It is to be understood that different sizes various elements, different numbers of shafts, different shaft shapes, and elements differing in various other ways may be used within the general scope of the present invention.)

A similar situation applies to the size, form, and thermal effectiveness of heat exchanger 650 of apparatus 500, as compared to heat exchangers made available in cryoprobes 104. The greater volume (per same diameter) available in apparatus 500 allows for a larger and more efficient heat exchanger 650 as compared to prior art heat exchangers available within probes 104. Since heat exchangers preferably provide a large surface of contact with flowing gasses, to enhance exchange of heat, they are necessarily both bulky and serve to impede, to some extent, flow of gasses around and through them. The advantages of apparatus 500 outlined above, in providing larger volume in gas input and exhaust lumens and enhanced inflow and outflow of gasses, enable to provide one or more highly efficient heat exchanger(s) 650 in apparatus 500.

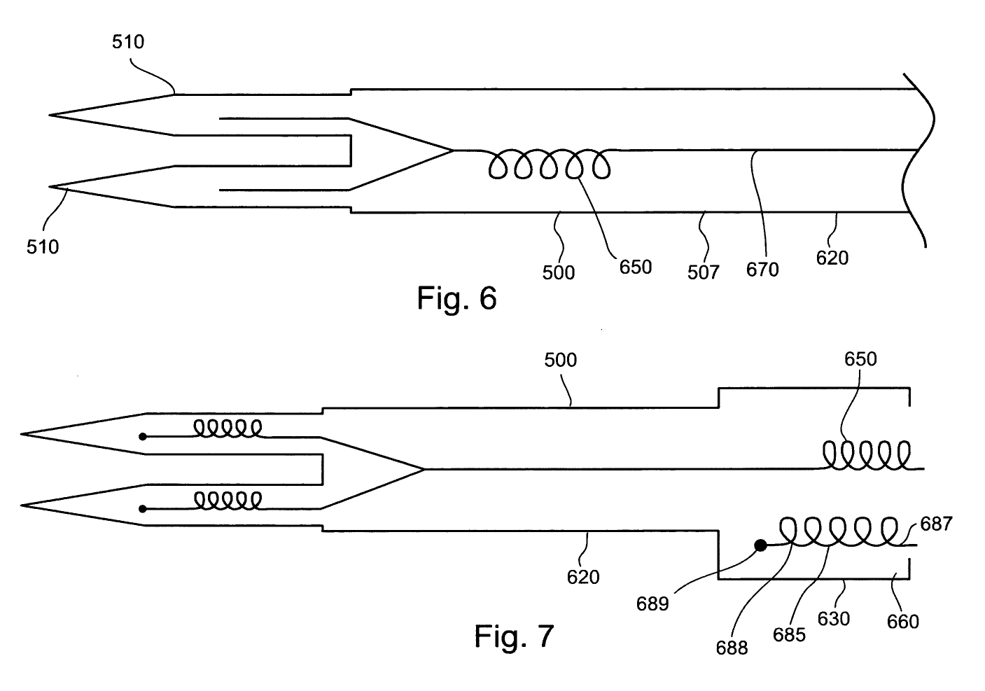

Attention is now drawn to FIG. 6, which is a simplified schematic of a cryoablation apparatus having a plurality of operating tips, which tips do not contain space-consuming heat exchangers, according to an embodiment of the present invention. FIG. 6 presents an apparatus 507 which is an embodiment of apparatus 500 having a plurality of operating tips 510, which operating tips 510 do not contain heat exchanger 550. As described in detail above, characteristics of apparatus 500 enable apparatus 500 to include a highly efficient proximal heat exchanger 650. Indeed, space available within shaft 620 enables heat exchanger 650 to be extremely large and/or enables to include a plurality of heat exchangers 650 within shaft 620. Accordingly, apparatus 500 may be constructed and will function successfully absent heat exchanging configurations 550 within operating tips 510. Absence of heat exchangers 550 within operating tips 510 enables operating tips 510 to be extremely thin, facilitating their penetration into cryoablation targets, and to be extremely flexible. Of course, reference here to “absence of heat exchangers 550” is not intended to imply that there may be no transfer of heat between operating tip gas input lumen 520 and tip exhaust passage 540. Operating tip gas input lumen 520 indeed may be constructed of thermally conductive metal so pre-cool high-pressure cooling gas prior to expansion. (Alternatively, operating tip gas input lumen 520 may be constructed of thermally non-conductive material such as plastic, to avoid heating of expanded cooling gas on its passage through tip exhaust passage 540.) In any case, apparatus 507 is characterized by lack of a space-consuming heat exchanger 550 within at least one and preferably all of operating tips 510. The term “space-consuming heat exchanger”, as used herein, refers to configurations of components which enhance heat exchange between those components by expanding shape or topological configuration of a component to increase contact surfaces between components. Thus, a substantially straight cryogen input lumen positioned within a substantially straight cryogen exhaust lumen will to some extent provide thermal transfer between input and output lumens, but is not a “space-consuming heat exchanger” as that term is used herein. However, if fins are provided on the walls of the input lumen, thereby enlarging surface of contact between those walls and gasses passing within the outer lumen, such a construction is “space-consuming heat exchanger” as that term is used herein. Similarly, configurations where one lumen is wrapped around another, or two lumens are spirally wrapped together, or where one lumen is presented in a spiral within another lumen, these and all similar configurations are “space-consuming heat exchangers” as that term is used herein.

Thus, in an embodiment shown in FIG. 6, operating tips 510 are characterized by having operating tip gas input lumen 520 formed as a straight conduit (i.e. not spirally coiled or otherwise turned or twisted or otherwise configured to increase surface of contact with gas transiting tip exhaust passage 540). Operating tip gas input lumen 520 is preferably straight and smooth. Operating tip gas input lumen 520 of apparatus 507 may, however, be flexible. Indeed absence of bulky space-consuming heat exchangers 550 allows operating tips 510 of apparatus 507 to be extremely thin, thus facilitating their penetration into an ablation target. Absence of heat exchangers 550 also allows operating tips 510 to be more flexible than tips which include heat exchangers, and to be flexible along all their length.

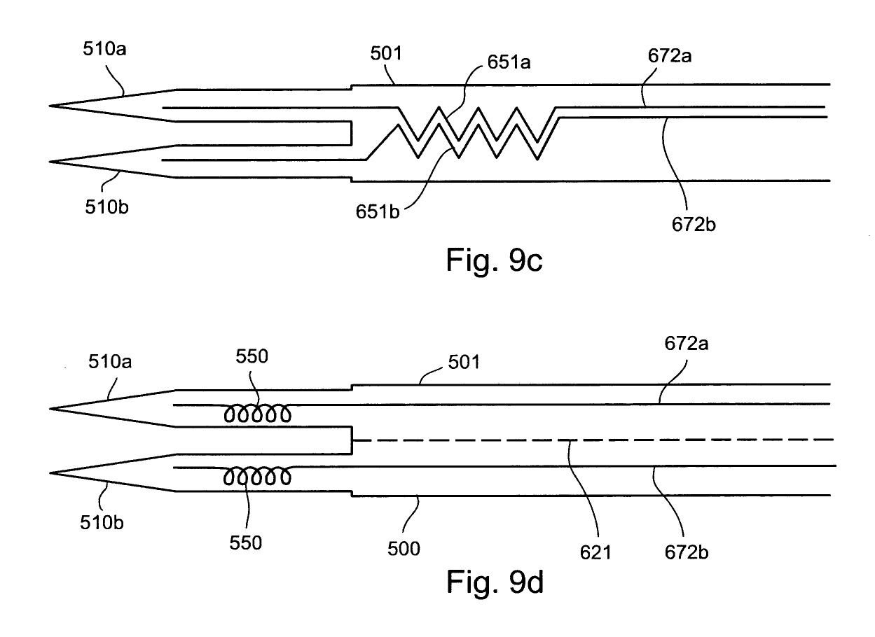

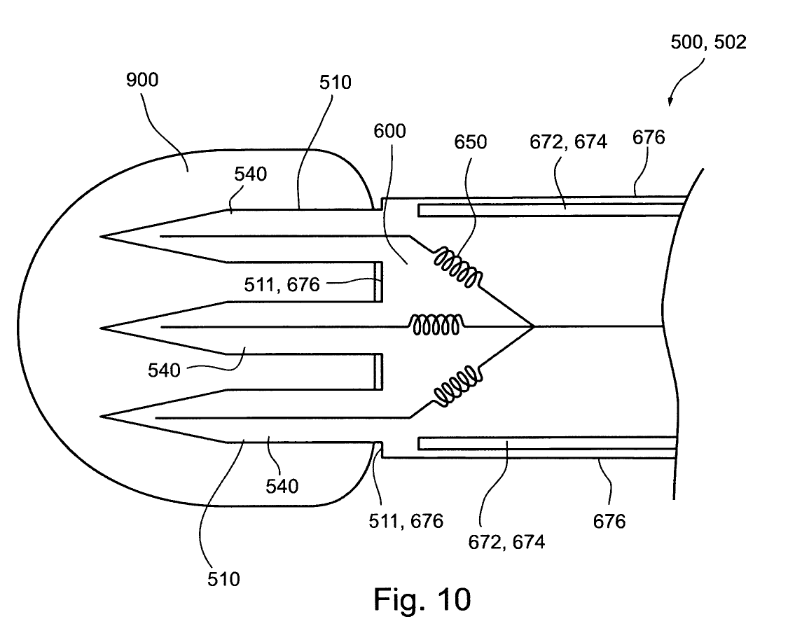

Thus, using design topology similar to that depicted in FIG. 6, and/or that discussed hereinbelow with respect to FIGS. 9a-9c and 10, the plurality of distal ends may be made very thin, for example 1.0 mm, 0.7 mm, 0.5 mm or less. Such thin distal ends may be made very flexible, and have the double advantage of being able to easily penetrate tissue and to cause less trauma to the tissues through which they pass, as compared to thicker probe ends of prior art.

It is noted that absence of heat exchangers 550 also enables manufacture of tips 510 in pre-bent configurations having greater curvature than that attainable in prior-art tips where heat exchangers are included in the tip.

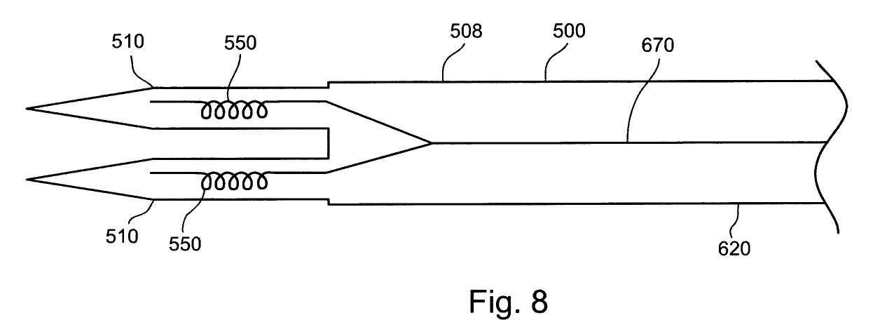

Attention is now drawn to FIG. 7, which is a simplified schematic of an embodiment of apparatus 500 which comprises an optional proximal pre-cooling unit, according to an embodiment of the present invention. Pre-cooling unit 685 may be positioned within shaft 620, preferably at a proximal portion of shaft 620, or in a wider proximal portion 660 of shaft 620, such as in a handle 630. Pre-cooling unit 685 preferably comprises an independent high-pressure gas input conduit 687, an heat exchanger 688 and a Joule-Thomson orifice 689, and serves to provide additional Joule-Thomson cooling (or, according to need, Joule-Thomson heating) in a proximal portion of apparatus 500, thereby contributing to pre-cooling (or pre-heating) of high-pressure gas input to operating tips 510. A head exchanger 650 may be positioned within or in proximity to pre-cooling unit 685, preferably enabling cold fluid from orifice 689 to flow over heat exchanger 650, thus further cooling high-pressure input gas. Alternatively, pre-cooling may be provided by evaporative cooling.

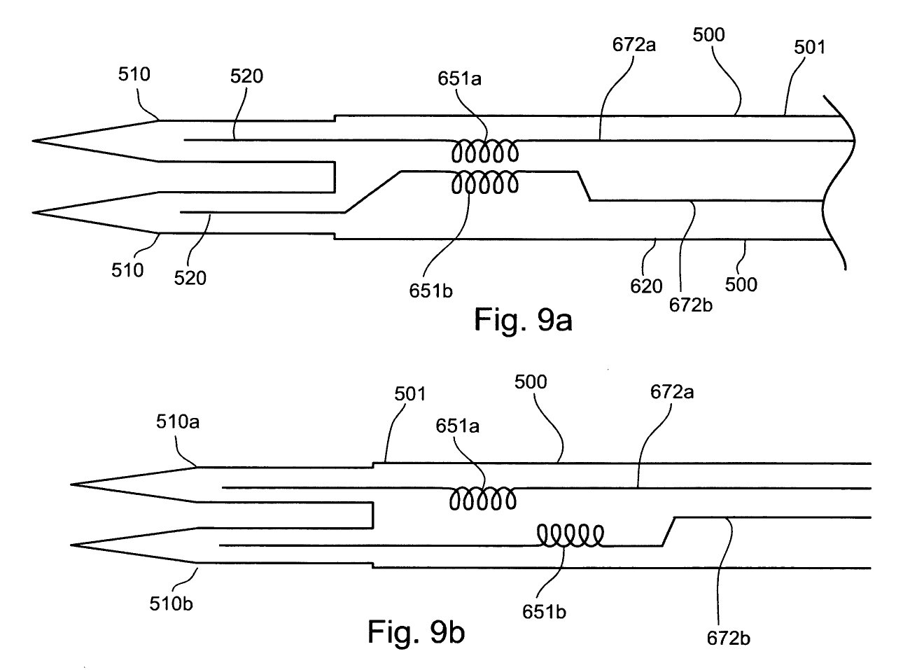

Attention is now drawn to FIG. 8, which is a simplified schematic presenting an alternative configuration of apparatus 500, here labeled apparatus 508, according to an embodiment of the present invention. As shown in FIG. 8, apparatus 500 may present heat exchangers 550 within each operating tip 510 and not comprise an additional space-consuming heat exchanger 650 within shaft 620. Note that this reference to an absence of heat exchangers 650 within a medial portion of this embodiment is not intended to imply that there may be no thermal transfer between medial gas input lumen 670 and medial exhaust lumen 610. Medial gas input lumen 670 may be constructed of thermally conductive material such as metal to enhance thermal transfer between incoming gas and exhaust gas. However, the embodiment presented in FIG. 8 is characterized by absence of a space-consuming heat exchanger enhancing heat exchange by expansion of surface of contact between input and output lumens. Thus, input lumen 670 of apparatus 508 is preferably constructed as a smooth straight conduit without twists or spirals or similar configurations.

Attention is now drawn to FIGS. 9a-9d, which are simplified schematics presenting alternative configurations of apparatus 500, here labeled apparatus 501. Apparatus 501 comprises a plurality of individual input gas conduits each individually supplying high-pressure gas to one of the plurality of operating tips 510, according to an embodiment of the present invention.

FIGS. 9a-9d present an apparatus 501 wherein gas supply to a plurality of operating tips 510 is individualized. In place of a common gas input lumen 760, a plurality of individual gas input conduits 672 are provided within shaft 620. Two such conduits, labeled 672a and 672b are shown in these exemplary Figures, but it is to be understood that this specific configuration is exemplary only and is not to be understood as limiting. Three, four, or more such conduits 672 may be provided. Optionally, each conduit 672 may be provided with an individual heat exchanger 651 (labeled 651a and 651b in these exemplary Figures). FIGS. 9a, 9b, 9c, and 9d are provided to demonstrate that individual conduit heat exchangers 651 may be positioned in parallel (FIG. 9a), in sequence (FIG. 9b), may be interleaved or intertwined (FIG. 9c) or may be entirely absent, preferably replaced by individual operating tip heat exchangers 550 (FIG. 9d).

Apparatus 501, by virtue of its individualized gas supply to each operating tip 510, may be used in a preferred mode of use whereby each operating tip 510 is individually connected to a high-pressure gas supply (not shown) in such a manner that gas supply may be started or stopped to each operating tip individually. According to methods well known in the art, a plurality of gas supply conduits 672 may be connected to a high-pressure gas supply through a system of valves enabling individual on-off control and preferably individual pressure control of gas supplied to each operating tip. Such a configuration enables a surgeon to control cooling of each operating tip 510 individually, and thereby enables the surgeon to adjust cooling of individual operating tips to tailor the size and shape of an ablation volume created by operating apparatus 501. Under such an arrangement it is even possible to operate one or more operating tips 510 in heating while operating one or more operating tips 510 in cooling, e.g. to control the shape of a ablation volume during cryosurgery. Since mixing hot and cold exhaust gasses in a common exhaust lumen would reduce efficiency of both heating and cooling processes, an additional embodiment designed for optional simultaneous heating and cooling is provided: one or more optional shaft subdividers 621 may be provided, as shown in FIG. 9d, to partially or wholly subdivide common shaft 620 into a plurality of gas exhaust lumens, thereby minimizing or eliminating interference between heating and cooling processes. Shaft subdividers 621 preferably comprise thermal insulating material. Since subdividers 621 divide separate lumens designed for low gas pressures within a common shaft, subdividers 621 do not require great mechanical strength and can be made of simple materials such as plastic.

Attention is now drawn to FIG. 10, which is a simplified schematic of a distal portion of an embodiment of apparatus 500, here labeled apparatus 502, according to an embodiment of the present invention. In apparatus 502, apparatus 500 has been configured so as to present a cooling surface along all or most of its distal face 511, in addition to cooling surfaces of operating tips 510. As has been noted hereinabove, expanded cooling gas exhausting from operating tips 510 is extremely cold. As has been further noted, back pressure in narrow gas exhaust lumens in prior art cryoprobes generally limits cooling of those probes because a pressure substantially higher than atmospheric pressure is maintained within gas exhaust lumens of typical cross-section. Operating tip exhaust passages 540 generally pose less resistance to passage of expanded exhaust gasses because of their relatively shorter length and optional absence of a heat exchanger, yet in most cases, unless exhaust passages 540 are relatively wide, a certain back pressure will develop therein during functioning of apparatus 502. Consequently, gas exiting exhaust passages 540 into relatively much wider medial portion 600 of apparatus 502 will undergo further decompression and consequent further cooling. Exhaust gas re-cooled by this further expansion will contribute substantially to pre-cooling of incoming gas by heat exchangers 650 within medial portion 600. Medial portion 600 functions in a manner similar to Joule-Thomson heater/cooler 200a within hollow 102 of prior art introducer 100 as shown in FIG. 3 and discussed hereinabove, in that Joule-Thomson decompression is used to cool a volume traversed by high-pressure gas input conduits. In contradistinction to that prior art configuration, however, additional cooling of medial portion 600 and consequent reinforced pre-cooling of incoming high-pressure cooling gas requires no additional high-pressure gas input sources: further decompression of exhaust gasses exiting gas exhaust passages 540 of operating tips 510 provides additional cooling during cooling phases of operation (and additional heating during heating phases of operation) by Joule-Thomson effect.

Intense cooling of medial portion 600 can be put to further use. Medial portion 600 may be cooled to such an extent that external walls 676 of medial portion 600 approach or reach cryoablation temperatures. In a preferred embodiment of apparatus 502, thermal insulation elements 672 or localized wall-heating elements 674 are provided to reduce thermal transfer to selected first portions of outside walls 676 of medial portion 600, whereas insulation and heating elements are absent from selected second portions of outside walls 676 of medial portion 600. In a preferred embodiment, insulated first selected portions of outside walls 676 are lateral walls of medial section 600, and uninsulated second selected portions of outside walls 676 of medial section 600 are distal face 511 of medial section 600, about and between positions where operating tips 510 extend distally from medial section 600. In a preferred method of use shown in FIG. 10, apparatus 502 is introduced into a body and extended so as to be facing a cryoablation target 900. Apparatus 502 is then displaced forward, causing a plurality of operating tips 510 to penetrate target 900, and preferably further causing distal face 511 of medial section 600 to be adjacent to and in thermal contact with target 900. Apparatus 502 is then operated in cooling, causing target 900 to be cooled both from without (by cold distal face 511) and from within (by cold operating tips 510), resulting in particularly rapid and effective cooling of target 900. Optionally, high-pressure heating gas is subsequently supplied to apparatus 502, causing target 900 to be warmed at all points of contact with apparatus 502, thereby causing melting of frozen tissues adhering to apparatus 502 and facilitating removal of the apparatus. It is noted that in FIG. 10 medial heat exchangers 650 are shown in individual gas conduits leading towards distinct operating tips 510, here shown without operating tip heat exchangers 550. In alternative constructions operating tip heat exchangers 550 may be present, and medial heat exchangers 650 may be absent and/or may be present on a common portion of high pressure gas input lumen, or individual high-pressure gas input conduits may be provided, with or without heat exchangers, for each operating tip 510.

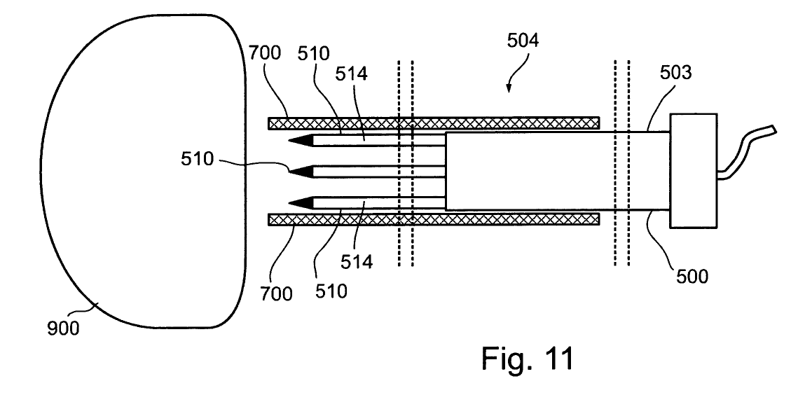

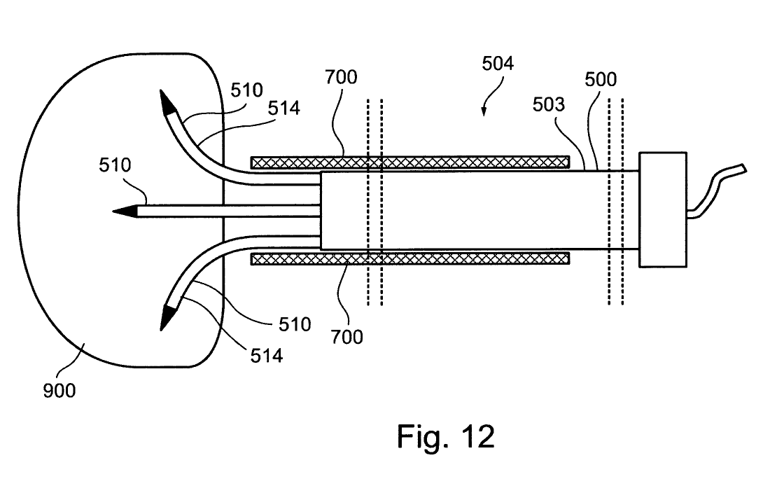

Attention is now drawn to FIGS. 11 and 12, which are simplified schematics of an apparatus 503 which comprises a multi-tipped cryoablation device combined with a tip-guiding sheath, according to an embodiment of the present invention. Apparatus 503 is any embodiment of apparatus 500 and is further characterized in that at least one of its operating tips 510 is a pre-bent tip 514. U.S. Pat. No. 6,706,037 to Zvuloni et al., and PCT Application IL2007/000091 have been discussed hereinabove and are incorporated herein by reference. These documents present in general the concept of a cryoprobe comprising nitinol or comprising pre-bent steel or other metal, and pre-formed, so that it may be constrained to be relatively straight, yet will assume a bent configuration when unconstrained and/or when brought to a pre-determined temperature. Apparatus 503 is characterized in that at least one of its plurality of operating tips is a pre-bent operating tip 514, preferably bent with an outward orientation, so pre-bent operating tip 514 assumes a lateral vector and bends away from the longitudinal axis of apparatus 504 when unconstrained. In a preferred embodiment, most or all operating tips of apparatus 503 are so formed, so that a plurality of operating tips 510 of apparatus 504 diverge from each other when unconstrained.

Apparatus 503 is preferably utilized with a tip-directing sheath 700. Apparatus 503 combined with sheath 700 are here labeled apparatus 504. Tip-directing sheath 700 performs as an introducer for apparatus 504. As shown in FIG. 11, apparatus 503 is preferably slightly withdrawn into sheath 700 when apparatus 504 is introduced into a body. Then, in a preferred mode of operation shown in FIG. 12, apparatus 503 is advanced within sheath 700, causing operating tips 510 to penetrate into a target. Operating tips 514 of apparatus 504, being pre-bent, diverge from each other as they escape the constraint of sheath 700, and tend to continue to diverge somewhat as they are inserted into an ablation target such as target 900. Operating tips 510/514, so divergent, are then well positioned to cryoablate a large ablation target, and in particular may be well positioned to ablate a target which is considerably broader than the diameter of apparatus 504.

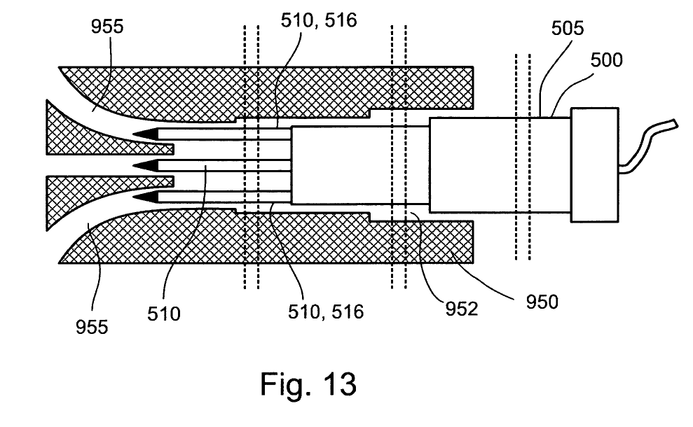

Attention is now drawn to FIG. 13, which is a simplified schematic of an additional embodiment of apparatus 500 comprising a tip insertion guide having a plurality of outwardly diverging tip channels, according to an embodiment of the present invention. U.S. Pat. No. 6,706,037 to Zvuloni et al., and PCT Application IL2007/000091, discussed hereinabove and incorporated herein by reference, have presented in general the use of an introducer having one or more a curved cryoprobe channels useable to guide movement of an flexible cryoprobe so as to cause a distal portion of a cryoprobe contained therein to assume a lateral vector when advanced beyond a distal end of that introducer channel. FIG. 13 presents an embodiment of apparatus 500 labeled apparatus 505 and characterized in that some or all of the plurality of operating tips 510 of apparatus 505 are flexible operating tips 516. FIG. 13 also presents a tip insertion guide 950 formed as a sheath having an internal lumen 952 sized to receive distal and medial portions of apparatus 506 therein, and further comprising a plurality of channels 955 each sized to accommodate an operating tip 510 of apparatus 506. In a preferred embodiment, some and preferably most of channels 955 are so curved as to constrain operating tips 516 to diverge as they advance through channels 955 and extend beyond channels 955. In a preferred mode of use, apparatus 505 is inserted in guide 950 and guide 950 is inserted in a body of a patient and caused to approach an ablation target 900. With a distal end of guide 590 adjacent to target 900, apparatus 505 is further advanced within guide 950, causing operating tips 510/516 to advance within channels 955, causing distal ends of operating tips 516 to diverge as they advance and penetrate target 900. Operating tips 510/516 are thus distanced one from another within target 900, and are thus well positioned to ablate a voluminous ablation target.

Guide 950 may have a sharp distal end to facilitate penetration into body tissue.

Guide 950 optionally comprises heat insulating material or a heater such as an electrical resistance heater.

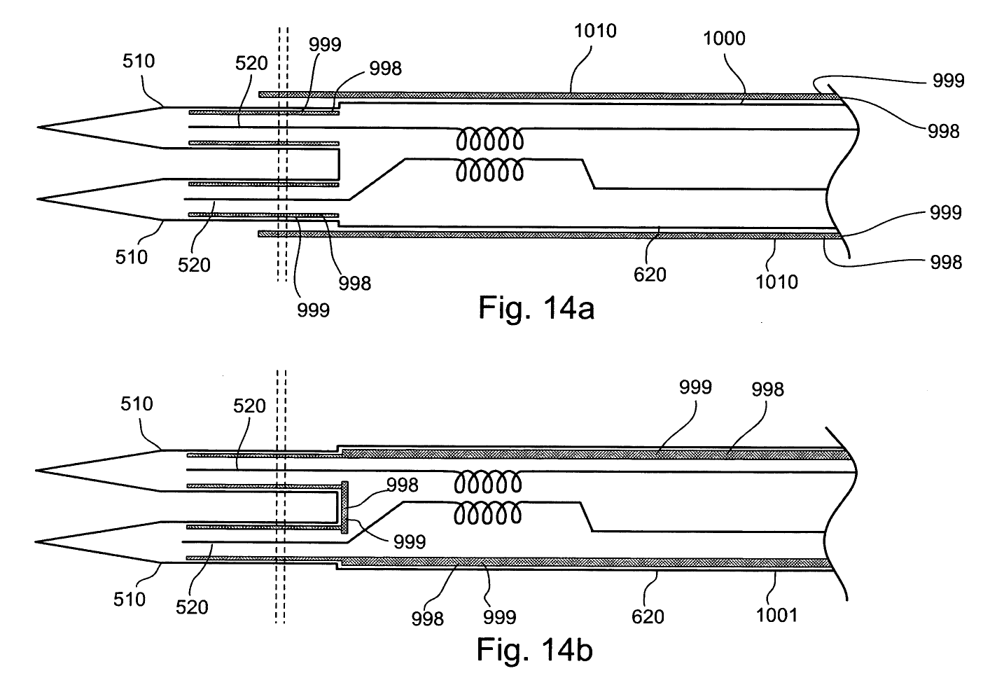

Attention is now drawn to FIGS. 14a and 14b, which are simplified schematics illustrating optional uses for heaters and/or thermal insulation in multi-headed cryoprobes, according to embodiments of the present invention.

FIGS. 14a and 14b present exemplary multi-headed cryoprobes labeled probe 1000 and probe 1001. A particular cryoprobe configuration has been selected for purposes of illustration, but it is be understood that the probe 1000 of FIG. 14a and probe 1001 of FIG. 14b should be understood to represent any of the embodiments and configurations presented in FIGS. 4-13 and discussed hereinabove, or any similar multi-headed cryoprobes.

FIG. 14a presents a probe 1000 wherein thermal insulation 999 is provided along lateral portions of operating tips 510. Probe 1000 is intended for use with an introducer 1010 which may also comprise thermal insulation 999. Alternatively, heating elements 998 may be provided in place of or in addition to thermal insulation 999 in tips 510 and/or in introducer 1010. Heating and/or insulation in introducer 1010 serves to protect tissues near shaft 620 of probe 1000, and therefore renders unnecessary heating/insulation along shaft 620.

FIG. 14b presents a probe 1001 wherein thermal insulation 999 and/or heating elements 998 are provided along shaft 620 and/or along selected portions of operating tips 510. Such thermal insulation and/or heating serves to protect tissues not intended for cryoablation from damage by cooled proximal portions of probe 1001.

In similarity to the embodiments presented by FIGS. 9a-9d and discussed hereinabove, embodiments presented by FIGS. 14a-14b, and in general any embodiments providing individualized cryogen supply to a plurality of operating tips, may be utilized in such a manner that different tips are differently activated.

For example, different tips may be used with different gas flow rates, creating a variation in tip heat removal capacities. Thus, should intense cooling or heating be required in one tip and not in others, that tip may be provided with full gas flow while other tips are used with reduced gas flow or with no gas flow. Such use will result in reduced back pressure within the common gas return lumen, and thereby strongly enhance the cooling capacity of the favored operating tip.

As noted above, various embodiments and in particular the embodiment presented by FIG. 9d may be used in a mode wherein one or more operating tips are operated using cooling gas while one or more other operating tips are operated using heating gas. It is to be noted that in such a case, if optional subdividers 621 are absent, exhausting heating gasses mix with exhausting cooling gasses in the common gas return lumen. Such mixing results in moderating of temperature of such exhausting gasses, and may, at appropriate mixtures, result in temperatures which will not cause thermal injury to adjacent tissues. Consequently, probes intended for use in this manner may be constructed with little or no thermal insulation lining the common shaft.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A cryotherapy apparatus comprising:
(a) a distal portion insertable in a body, said distal portion comprises a plurality of operating tips, each comprises a distal cryogen expansion chamber; and
(b) a medial portion which comprises a medial cryogen exhaust lumen communicating with at least two of said distal cryogen expansion chambers.

2. The cryotherapy apparatus of claim 1, wherein each of said operating tips further comprises a distal cryogen input lumen, and said medial portion further comprises a medial cryogen input lumen communicating with at least one of said distal cryogen input lumens.

3. The apparatus of claim 2, wherein at least one of said operating tips comprises a Joule-Thomson cooler.

4. The apparatus of claim 2, further comprising a medial heat exchanger operable to facilitate exchange of heat between said medial cryogen input lumen and said medial cryogen exhaust lumen.

5. The apparatus of claim 2, wherein at least one of said operating tips further comprises a distal cryogen exhaust lumen and a distal heat exchanger which facilitates heat exchange between said distal cryogen exhaust lumen and said distal cryogen input lumen.

6. The apparatus of claim 2, wherein said medial portion is configured for insertion in a body.

7. The apparatus of claim 2, further comprising a proximal portion sized and configured to remain outside a body when said distal portion is inserted in a body.

8. The apparatus of claim 2, further comprising
(a) a proximal portion which comprises a first proximal cryogen input lumen which communicates with said medial cryogen input lumen;
(b) a proximal cryogen exhaust lumen which communicates with said medial cryogen exhaust lumen; and
(c) a heat exchanger which facilitates exchange of heat between said proximal cryogen input lumen and said proximal cryogen exhaust lumen.

9. The apparatus of claim 8, wherein said proximal portion comprises a second proximal cryogen input lumen distinct from said first proximal cryogen input lumen, and a cryocooler communicating with said second proximal cryogen input lumen.

10. The apparatus of claim 9, wherein said cryocooler is a Joule-Thomson cooler.

11. The apparatus of claim 2, wherein at least one of said operating tips is so configured that said distal gas input lumen is formed as a conduit within said distal exhaust lumen and said conduit is substantially straight.

12. The apparatus of claim 11, wherein at least one of said operating tips is substantially flexible.

13. The apparatus of claim 11, wherein a maximum outer diameter of at least one of said operating tips is less than 1.0 mm.

14. The apparatus of claim 11, wherein outer diameter of at least one of said operating tips is less than 0.7 mm.

15. The apparatus of claim 11, wherein a maximum outer diameter of at least one of said operating tips is less than 0.5 mm.

16. The apparatus of claim 2, comprising a space-consuming heat exchanger only in said medial portion.

17. The apparatus of claim 2, comprising a space-consuming heat exchanger only in said proximal portion.

18. The apparatus of claim 2, comprising space-consuming heat exchangers only in said medial and proximal portions.

19. The apparatus of claim 2, wherein said distal cryogen input lumen and said distal cryogen exhaust lumen are uncoiled.

20. The apparatus of claim 2, wherein said distal cryogen input lumen and said distal cryogen exhaust lumen are of substantially similar length.

21. The apparatus of claim 2, wherein said medial cryogen input lumen is positioned within said medial cryogen exhaust lumen and is substantially straight.

22. The apparatus of claim 2, wherein said medial portion does not contain a space-consuming heat exchanger.

23. The apparatus of claim 2, wherein said medial cryogen input lumen is operable to transport high-pressure gas to at least two of said distal gas input lumens.

24. The apparatus of claim 2, further comprising a plurality of medial cryogen input lumens each operable to transport high-pressure gas to a one of said plurality of distal cryogen input lumens.

25. The apparatus of claim 24, further comprising a plurality of heat exchangers, each operable to facilitate transfer of heat between input cryogen in one of said medial cryogen input lumens and exhaust cryogen within said medial cryogen exhaust lumen.

26. The apparatus of claim 2, wherein a least a section of a lateral wall of said medial portion is insulated and a distal face of said medial portion is uninsulated.

27. The apparatus of claim 2, so configured that high-pressure input gas provided at a Joule-Thomson orifice within at least a one of said plurality of operating tips will undergo a first expansion within said operating tip and a subsequent second expansion when passing from said operating tip into said medial portion.

28. The apparatus of claim 26, so configured that high-pressure input gas provided at a Joule-Thomson orifice within at least a one of said plurality of operating tips will undergo a first expansion within said operating tip and a subsequent second expansion when passing from said operating tip into said medial portion.

29. The apparatus of claim 2, wherein at least one of said operating tips is pre-bent.

30. The apparatus of claim 29, wherein a plurality of said operating tips are pre-bent.

31. The apparatus of claim 30, wherein said plurality of pre-bent operating tips are so configured and so oriented that distal ends of said operating tips diverge from each other when said operating tips are unconstrained.

32. The apparatus of claim 31, further comprising a tip-guiding sheath sized to accommodate said distal portion and at least a part of said medial portion, which sheath is operable to limit divergence of said distal ends of said operating tips when said distal portion is contained within said sheath.

33. The apparatus of claim 2, further comprising a tip insertion guide having a plurality of outwardly diverging tip channels.

34. A cryotherapy apparatus which comprises a plurality of distal operating tips connected to a common medial shaft.

35. The apparatus of claim 34, wherein said common shaft comprises at least one shaft subdivider which at least partially subdivides said medial shaft into a plurality of gas exhaust lumens.

36. A method of treating an organic cryotherapy target within a body of a patient, which comprises
(a) providing a cryotherapy apparatus which comprises
(a) a distal portion insertable in a body, said distal portion comprises a plurality of operating tips, each comprises
(i) a distal cryogen input lumen; and
(ii) a distal cryogen expansion chamber; and
(b) a medial portion which comprises
(i) a medial cryogen input lumen communicating with at least one of said distal cryogen input lumens; and
(ii) a medial cryogen exhaust lumen communicating with at least two of said distal cryogen expansion chambers;
(b) inserting said plurality of operating tips into said organic cryotherapy target within a body of a patient; and
(c) supplying a cryogen to said plurality of expansion chambers via said medial and distal cryogen input lumens,
thereby cooling said plurality of distal operating tips, thereby treating said cryotherapy target.

37. A method of treating an organic cryotherapy target within a body of a patient, comprising
(a) providing a cryotherapy apparatus which comprises a plurality of operating tips and a common cryogen exhaust lumen;
(b) supplying high-pressure gas to at least two of said operating tips.

38. The method of claim 37, further comprising inserting said plurality of operating tips into said organic cryotherapy target, then supplying said gas to said at least two operating tips.
